(19) United States
(12) Patent Application Publication
(10) Pub. No.: US 2016/0324424 A1
(43) Pub. Date: Nov. 10, 2016

(54) DETERMINING TEMPERATURE WITH THE AID OF AN INTERVENTION APPARATUS

(71) Applicants: Arne Hengerer, Mohrendorf (DE); Volker Matschl, Bamberg (DE); Martin Requardt, Nurnberg (DE); Rainer Schneider, Erlangen (DE)

(72) Inventors: Arne Hengerer, Mohrendorf (DE); Volker Matschl, Bamberg (DE); Martin Requardt, Nurnberg (DE); Rainer Schneider, Erlangen (DE)

(21) Appl. No.: 15/146,976

(22) Filed: May 5, 2016

(30) Foreign Application Priority Data
May 6, 2015 (DE) 102015208420.5

(51) Int. Cl. A61B 5/01; G01K 13/00; A61B 18/14; A61B 5/055; A61B 5/06; A61B 34/20; G01R 33/28; A61B 5/00

(52) U.S. Cl. CPC A61B 5/01; G01R 33/286; G01R 33/288; G01K 13/002; A61B 5/6847; A61B 5/7425; A61B 5/055; A61B 5/06; A61B 34/20; A61B 5/704; A61B 18/1492; A61B 2034/2051; A61B 2018/00577

(57) ABSTRACT

System and methods are provided for determining temperature with the aid of an intervention apparatus. A reference temperature at a detection location inside the examination object is detected using a reference temperature measuring unit. A relative temperature distribution in a measuring volume, having a spatial position in relation to the detection location of the reference temperature that is known, is detected using a temperature distribution measuring unit. An evaluation unit ascertains an absolute temperature distribution using the reference temperature and the relative temperature distribution.

### CROSS-REFERENCE TO RELATED APPLICATIONS

This application claims the benefit of DE 10 2015 20 84 20.5, filed on May 6, 2015, which is hereby incorporated by reference in its entirety.

### TECHNICAL FIELD

Embodiments relate to determining temperature.

### BACKGROUND

Imaging methods are important aids in medical engineering. Magnetic resonance imaging (MRI) is therefore characterized by high and variable soft tissue contrasts in clinical slice imaging for instance. Fast-switched gradient fields are superimposed on a static basic magnetic field in a magnetic resonance device. The fast-switched gradient fields are generated by a gradient system of the magnetic resonance device. High-frequency (HF) pulses are irradiated into the examination object by a high-frequency antenna unit of the magnetic resonance device in order to trigger magnetic resonance signals. The magnetic resonance signals triggered thereby are recorded, and based on this, magnetic resonance images are created.

Interventions, such as, for example, ablations, may be supported by MRT imaging since MRT imaging enables straightforward navigation of a catheter. For example, disruptions to the conductivity in the heart, such as atrial fibrillation or ventricular fibrillation, may be treated (e.g., by annular ablation around the pulmonary vein) using targeted ablation of specific areas of the heart muscle (e.g., myocardium). A catheter is conventionally introduced into the heart for this purpose, and, for example, high-frequency energy is locally applied for the purpose of tissue ablation.

According to the prior art, catheter intervention takes place with fluoroscopic control.

### SUMMARY AND DESCRIPTION

The scope of the present invention is defined solely by the appended claims and is not affected to any degree by the statements within this summary. The present embodiments may obviate one or more of the drawbacks or limitations in the related art.

It would be desirable in the connection to be able to control the quality (e.g., the transmurality) of an ablation and/or potential ablation process, such as, for example, a perforation of the myocardium and/or excessive heating of the adjoining esophagus, directly during the intervention. However, the transmurality of an ablation may not be unequivocally verified by using implementable techniques. The success of the intervention may only be clinically determined in the weeks thereafter with the aid of symptoms.

The present embodiments may obviate one or more of the drawbacks or limitations in the related art. For example, a method and a device that enable better control during an intervention are provided.

According to an embodiment, a method for determining temperature with the aid of an intervention apparatus inside an examination object is provided. The method includes detecting a reference temperature at a detection location inside the examination object using a reference temperature measuring unit. A relative temperature distribution in a measuring volume, with a spatial position in relation to the detection location of the reference temperature that is known, is detected using a temperature distribution measuring unit. An absolute temperature distribution is ascertained by an evaluation unit using the reference temperature and relative temperature distribution.

The intervention apparatus may be, for example, a catheter, such as an ablation catheter. The intervention apparatus is located inside the examination object at the time the method is carried out. The examination object may be, for example, a human or animal body (e.g., a heart of a human or a heart of an animal body).

The relative temperature distribution may be an allocation of temperature values relating to spatial coordinates that are located in the measuring volume. The relative temperature distribution may be displayed by a temperature map. The relative temperature distribution may include only information about temperature differences between the locations that are represented by the coordinates.

By detecting the reference temperature, the detected relative temperature distribution may be standardized (e.g., using the evaluation unit). For example, the reference temperature may produce a reference between the relative temperature distribution and the absolute temperature distribution. Mathematically the reference may be formulated, for example, as follows: T_{abs}(r)=T_{ref}(R)−Tᵣₑₗ(R)+Tᵣₑₗ(r). T_{abs}(r) is the absolute temperature, and Tᵣₑₗ(r) is the relative temperature at location r. Since a relative temperature is detected for a plurality of locations r inside the measuring volume V, with a spatial position in relation to the detection location of the reference temperature that is known, the relative temperature distribution results herefrom. T_{ref}(R) is the reference temperature, and Tᵣₑₗ(R) is the relative temperature at detection location R. Accordingly, detection location R is advantageously incorporated by measuring volume V in order to be able to determine the relative temperature Tᵣₑₗ(R). The relative temperature distribution may be standardized to the reference temperature by the term T_{ref}(R)−Tᵣₑₗ(R). A clear relationship between r and R is also given by the known spatial position of the measuring volume V, and therewith the locations r incorporated thereby, in relation to detection point R.

Without the standardization, only relative temperature values having substantially lower significance may be used to assist an intervention. The method of one or more of the present embodiments, by contrast, enables a quantitative temperature measurement (e.g., inside a heart muscle and adjoining risk structures) without a reference having to be recorded in advance. The ascertained absolute temperature distribution may advantageously be used to assist when carrying out an intervention since the ascertained absolute temperature distribution enables conclusions about the condition and/or a manipulation of a tissue that is incorporated by the examination object. The measuring volume is advantageously located in a spatial region that may be influenced by an intervention.

A reference temperature signal at the reference location inside the examination object is may be measured by the reference temperature measuring unit of the intervention apparatus in order to detect the reference temperature. The measured reference temperature signal is transmitted to the evaluation unit.

The reference temperature measuring unit is therefore incorporated by the intervention apparatus that is positioned inside the examination object, so the reference temperature measuring unit is arranged in the vicinity of a potential intervention. The positioning enables easy and accurate measurement of the reference temperature. The reference temperature signal may be forwarded to the evaluation unit via one or more supply line(s) and interfaces.

A temperature distribution signal from the measuring volume inside the examination object is measured by a temperature distribution measuring unit of the intervention apparatus in order to detect the relative temperature distribution. The measured temperature distribution signal is transmitted to the evaluation unit.

The temperature distribution measuring unit is therefore incorporated by the intervention apparatus. The temperature distribution signal may be, for example, a magnetic resonance signal. Since the temperature distribution measuring unit is incorporated by the intervention apparatus and has compact dimensions, one or more micro coil(s) are primarily suitable for recording the magnetic resonance signal from which the relative temperature distribution may be derived using methods that are known from MR thermometry. The relative temperature distribution is detected by a measurement of a phase difference and/or a diffusion and/or a T1 relaxation and/or a T2 relaxation and/or a proton resonance frequency (PRF) and/or a proton density.

Even further methods for detecting the relative temperature distribution may be known to a person skilled in the art. The methods may be based, for example, on an image difference (e.g., at least two MR images are detected in a time-resolved manner, where a first of the at least two MR images serves as a reference for at least one more of the at least two MR-images). Reference-free and/or reference-less methods may also be provided.

The ascertained absolute temperature distribution is provided, or displayed, for example, using a display unit of a magnetic resonance device. A person involved in a potential intervention may be provided with information via such a display. The display may be used to carry out the potential intervention. For example, a particular continuously updated display of color-coded temperature maps may be provided. A temperature map may depict, for example, the absolute temperature distribution in a cutting plane of the detected measuring volume.

One embodiment provides that a feedback signal is output when a feedback condition exists as a function of the ascertained absolute temperature distribution.

The feedback signal may warn a person involved in carrying out an intervention about potentially dangerous situations. The feedback signal may be, for example, an acoustic and/or haptic and/or visual signal.

The ascertained absolute temperature distribution includes at least one absolute temperature value. A feedback signal is output when a threshold value is exceeded by the at least one absolute temperature value. For example, a transmural ablation and/or a risk of a myocardial perforation may be correlated with exceeding a specific temperature threshold.

The position of the intervention apparatus inside the examination object is determined. The position determination of the intervention apparatus inside the examination object may also be referred to as tracking. A distinction is made between active and passive tracking. Passive tracking is based on image artifacts or image properties that are created by the intervention apparatus.

Active tracking uses a device (e.g., a localization unit) that may receive a position-dependent signal. A signal (e.g., a high-frequency signal) may be generated by a magnetic resonance sequence or by separate signal generators. The signal may be directly detected. Hand detection may occur indirectly using magnetic resonance signals. Nuclei are resonantly excited to the Larmor frequency. The Larmor frequency is the frequency with which the spins of the nuclei are in precession about the direction of the outer magnetic field. The nuclei emit the excitation energy again via irradiation of a magnetic resonance signal. Since the magnetic resonance signal conventionally has location information, the magnetic resonance signal may be utilized for active tracking.

A localization signal (e.g., an HF signal) or a magnetic resonance signal may be measured by a localization unit of the intervention apparatus in order to determine the position of the intervention apparatus. The localization signal is transmitted to the evaluation unit. The localization signal contains information, using which the evaluation unit may carry out the position determination of the intervention apparatus.

The localization unit is therefore incorporated by the intervention apparatus. For example, a suitable localization unit may have one or more HF coil(s) that are arranged on and/or in the intervention apparatus to allow active tracking.

In an embodiment, the position determination of the intervention apparatus is assisted by roadmap images. Using a preceding measurement, one or more roadmap image(s) of at least part of the examination object in which the position of the intervention apparatus is shown are therefore generated. The roadmap image facilitates orientation inside the examination object.

In an embodiment, an image of at least part of the examination object is generated. For example, an image potentially does not map, for example, an entire person but only part of his cardiac muscle. The mapped part advantageously at least partly includes the measuring volume of the detected relative temperature distribution.

An image data signal is detected by an image data acquisition unit of the intervention apparatus and/or a further apparatus. The image data signal is transmitted to the evaluation unit for generation of the image of at least part of the examination object. As an alternative to or in addition to an image data acquisition unit, which is incorporated by the intervention apparatus, an external image data acquisition unit, such as, for example, a local coil that is arranged on the surface of the examination object, may record an image data signal. In one embodiment, image data signals from an image data acquisition unit incorporated by the intervention apparatus may be combined with an image data acquisition unit incorporated by the further apparatus to form an image. For example, the signal of a micro coil, used as an image data acquisition unit in the intervention apparatus, may be combined with a morphological image of an external, larger coil, for example, in that the image of the micro coil is superimposed on the image of the larger coil.

The evaluation unit is configured to generate an image using the image data signal. The image provides a person involved in an intervention with information, for example, on a screen. The provided image is continuously provided, so a current image exists. The absolute temperature distribution and/or the position of the intervention apparatus may also be displayed in the image, via which, for example, a tissue in which an intervention is potentially made is displayed.

In an embodiment, the image data acquisition unit may be incorporated by the temperature distribution measuring unit and/or the localization unit. For example, the temperature distribution signal and/or the localization signal may be identical to the image data signal. For example, in the case of magnetic resonance signals as a temperature distribution signal and/or localization signal, the temperature distribution signal and/or localization signal may be used to generate images.

In an embodiment, the temperature distribution measuring unit may be incorporated by the localization unit. For example, the temperature distribution signal may be identical to the localization signal. For example, in the case of magnetic resonance signals as a temperature distribution signal, the magnetic resonance signals may be used for position determination of the intervention apparatus.

The method for determining temperature of one or more of the present embodiments may be carried out at the same time as and/or after (e.g., alternately to), a potential intervention. An intervention itself may include, for example, an ablation (e.g., of a myocardial area). The temperature distribution may only be in projection of the intervention apparatus (e.g., the catheter).

The ascertained absolute temperature distribution and/or a localized position of the intervention apparatus and/or the generated image of at least part of the examination object is particularly jointly provided (e.g., displayed). The display enables clear and convenient monitoring of a potential intervention and/or navigation of the intervention apparatus (e.g., a catheter).

During detection of the reference temperature and the relative temperature distribution, the intervention apparatus may rest on a moving organ. A disruptive relative movement between the intervention apparatus (e.g., the reference temperature measuring unit and/or the temperature distribution measuring unit and/or the image data acquisition unit) and the moving organ, such as, for example, a myocardium, may be avoided thereby.

Embodiments of an intervention apparatus will be described below. The advantages thereof substantially match the advantages of the method for determining temperature with the aid of the intervention apparatus, which have been stated above in detail. Features, advantages or alternative embodiments mentioned in this connection may also be transferred to the other subject matters and vice versa.

An intervention apparatus includes an intervention unit, a reference temperature measuring unit for detecting a reference temperature signal, and a temperature distribution measuring unit for detecting a temperature distribution signal. A reference temperature may be determined using the reference temperature signal, and a relative temperature distribution may be determined using the temperature distribution signal.

The intervention unit may be, for example, a catheter (e.g., an ablation catheter).

One embodiment of the intervention apparatus provides that the intervention unit is configured to carry out an ablation (e.g., a high-frequency ablation and/or cryoablation, of an organic tissue, such as a myocardium).

The intervention unit may be, for example, an ablation unit, such as an HF electrode and/or a cryo electrode.

During cryoablation, liquid coolant, such as, for example, nitrogen oxide and/or nitrous oxide, is conveyed into a hollow tip of the ablation catheter. The liquid coolant evaporates there and removes heat from the surrounding tissue. Tissue cells may be destroyed by the formation of ice.

A high-frequency alternating current is conventionally conducted through an HF electrode (e.g., inductivity in the form of a coil), whereby heat is produced at the location of the HF electrode. The heat may be fed to surrounding tissue cells, so the surrounding tissue cells may be destroyed.

The intervention unit is arranged at the end of the intervention apparatus (e.g., the intervention unit is located at one end and/or a tip of the intervention apparatus). The intervention unit may be used particularly effectively as a result.

One embodiment of the intervention apparatus provides that the temperature distribution measuring unit is configured to measure a temperature distribution signal. The temperature distribution measuring unit includes at least one HF coil that may be configured as a micro coil. The HF coil is configured to receive magnetic resonance signals. The received magnetic resonance signals may be used to apply an MR thermometry method.

In one embodiment, the temperature distribution measuring unit includes a measuring volume that includes an extension parallel to a longitudinal axis of the intervention apparatus of a myocardium thickness (e.g., 30 mm, 20 mm, or 15 mm). The transmurality of an ablation may be assessed reliably. The myocardium thickness is the thickness of a cardiac muscle.

An HF coil as a temperature distribution measuring unit that is configured such that detection of a temperature distribution beyond the range of a myocardium thickness is located upstream of the catheter tip may be provided. The HF coil is located, for example, on an ablation catheter as far as possible in the immediate vicinity of an ablation electrode. Since a catheter is conventionally tubular, the longitudinal axis of the catheter is the center line and/or axis of symmetry of the tubular shape.

One embodiment of the intervention apparatus provides that a temperature at a reference location inside the examination object may be ascertained using the detected reference temperature signal. The reference location is advantageously located inside the measuring volume of the temperature distribution measuring unit. The reference temperature measuring unit may be provided and/or formed on a tip of the intervention apparatus to measure a temperature directly at the tip of the intervention apparatus. Using the reference temperature signal, the relative temperature distribution detected using the temperature distribution measuring unit may be standardized, so that an absolute temperature distribution may be ascertained therefrom.

The reference temperature measuring unit includes a temperature sensor, such as, for example, a thermoelement and/or a resistance-based temperature sensor. Temperature sensors have good availability.

One embodiment of the intervention apparatus provides that the intervention apparatus also includes a localization unit. The position and/or situation of the intervention apparatus inside the examination object may be ascertained using the localization unit (e.g., the intervention apparatus may be tracked). The localization unit is configured to receive a localization signal that may be supplied to an evaluation unit.

The localization unit may include at least one HF coil that is configured to receive an HF signal. Active tracking may advantageously be carried out using the at least one HF coil. If the localization unit includes two or more coils, an orientation of the intervention apparatus may also be determined in addition to a position. In the variant that the coil is only provided at the end of the intervention apparatus, tracking of the intervention apparatus using roadmap images may also be provided.

One embodiment of the intervention apparatus provides that the intervention apparatus includes an image data acquisition unit. The image data acquisition unit is configured to detect an image data signal (e.g., a magnetic resonance signal) using the images of which at least some of the examination object may be generated.

The units incorporated by the intervention apparatus have separate supply lines that include blocking circuits. The blocking circuits may prevent high-frequency displacement currents that may lead to undesirable heating of the examination object from being induced on the supply lines by a high-frequency antenna unit of a magnetic resonance device.

In an embodiment, the intervention apparatus includes an interface that is configured to connect the intervention apparatus to an evaluation unit for transmitting signals. The interface may be connected to the units incorporated by the intervention apparatus via supply lines to these units. The interface may be configured, for example, in the form of a plug-in connection.

A magnetic resonance device having an evaluation unit is provided. The magnetic resonance device includes an intervention apparatus. The evaluation unit is configured to ascertain an absolute temperature distribution using a reference temperature signal and a temperature distribution signal. The evaluation unit may include a memory and/or one or more processors. For example, the evaluation unit is configured to ascertain a reference temperature from the reference temperature signal and/or to ascertain a relative temperature distribution from the temperature distribution signal.

A computer program product that includes a program and may be loaded directly into a memory (e.g., a non-transitory computer-readable storage medium) of a programmable system control unit of a magnetic resonance device is provided. The computer program product includes program code (e.g., instructions; libraries and auxiliary functions) to carry out a method for determining temperature when the computer program product is run in the system control unit of the magnetic resonance device.

The computer program product may include software with a source code that has still to be compiled and embedded or that only is to be interpreted, or an executable software code that for execution only is to be loaded in the system control unit. The method may be carried out by the computer program product quickly, robustly, and in a manner that may be repeated in an identical manner. The computer program product is configured such that the computer program product may carry out the method acts using the system control unit. The system control unit includes, for example, a main memory, a graphics card, or a logic unit. The computer program product is stored, for example, on a computer-readable medium (e.g., a non-transitory computer-readable storage medium) or on a network or server, from where the computer program product may be loaded into the processor of a local system control unit that may be directly connected to the magnetic resonance device or be designed as part of the magnetic resonance device. Control information of the computer program product may also be stored on an electronically readable data carrier. The control information of the electronically readable data carrier may be configured such that the control information carries out a method when the data carrier is used in a system control unit of the magnetic resonance device. Examples of electronically readable data carriers are a DVD, magnetic tape, or a USB stick, on which electronically readable control information (e.g., software) is stored. When the control information is read from the data carrier and stored in a system control unit of the magnetic resonance device, all embodiments of the methods described above may be carried out. An embodiment may therefore also start from the computer-readable medium and/or the electronically readable data carrier.

### BRIEF DESCRIPTION OF THE DRAWINGS

Further advantages, features and details of the invention emerge from the exemplary embodiments described below and with reference to the drawings. Mutually corresponding parts are provided with the same reference numerals in all figures.

FIG. 1 depicts an example schematic diagram of a magnetic resonance device.

FIG. 2 depicts an example schematic diagram of an intervention apparatus.

FIG. 3 depicts a flowchart of an example method for determining temperature with the aid of the intervention apparatus.

FIG. 4 depicts an expanded flowchart of an example method for determining temperature with the aid of the intervention apparatus.

### DETAILED DESCRIPTION

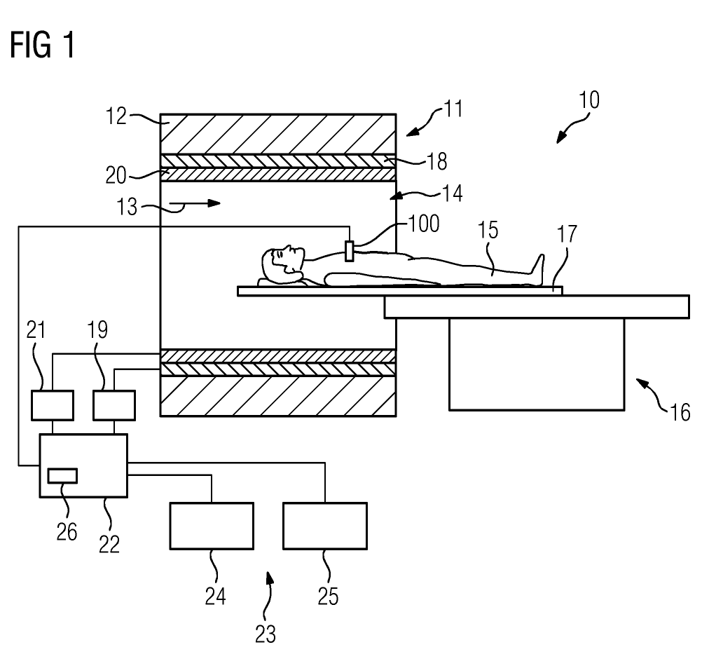

FIG. 1 schematically depicts a magnetic resonance device 10. The magnetic resonance device 10 includes a magnetic unit 11 that includes a superconductive main magnet 12 for generating a strong and, for example, main magnetic field 13 that is constant over time. The magnetic resonance device 10 also includes a patient-receiving region 14 for receiving a patient 15. In the present exemplary embodiment, the patient-receiving region 14 is cylindrical and cylindrically surrounded by the magnetic unit 11 in a circumferential direction. A different design of the patient-receiving region 14 may, however, be provided. The patient 15 may be pushed using a patient-positioning device 16 of the magnetic resonance device 10 into the patient-receiving region 14. The patient-positioning device 16 has an examination table 17 arranged so as to move inside the patient-receiving region 14.

The magnetic unit 11 also has a gradient coil unit 18 for generating magnetic field gradients that are used for spatial encoding during imaging. The gradient coil unit 18 is controlled using a gradient control unit 19 of the magnetic resonance device 10. The magnetic unit 11 also includes a high-frequency antenna unit 20 that in the present exemplary embodiment is configured as a body coil permanently integrated in the magnetic resonance device 10. The high-frequency antenna unit 20 is configured for an excitation of nuclei, which is established in the main magnetic field 13 generated by the main magnet 12. The high-frequency antenna unit 20 is controlled by a high-frequency antenna control unit 21 of the magnetic resonance device 10 and irradiates high-frequency magnetic resonance sequences into an examination space that is substantially formed by a patient-receiving region 14 of the magnetic resonance device 10. The high-frequency antenna unit 20 is also configured to receive magnetic resonance signals.

For controlling the main magnet 12 of the gradient control unit 19 and for controlling the high-frequency antenna control unit 21, the magnetic resonance device 10 has a system control unit 22 (e.g., system controller). The system control unit 22 centrally controls the magnetic resonance device 10, such as, for example, carrying out a predetermined imaging gradient echo sequence. The system control unit 22 includes an evaluation unit (not shown) for evaluation of medical image data that is acquired during the magnetic resonance examination. The magnetic resonance device 10 also includes a user interface 23 that is connected to the system control unit 22. Control information, such as, for example, imaging parameters and reconstructed magnetic resonance images may be displayed on a display unit 24 (e.g., on at least one monitor) of the user interface 23 for a medical operator. The user interface 23 also has an input unit 25 used for inputting information and/or parameters by the medical operator during a measuring process.

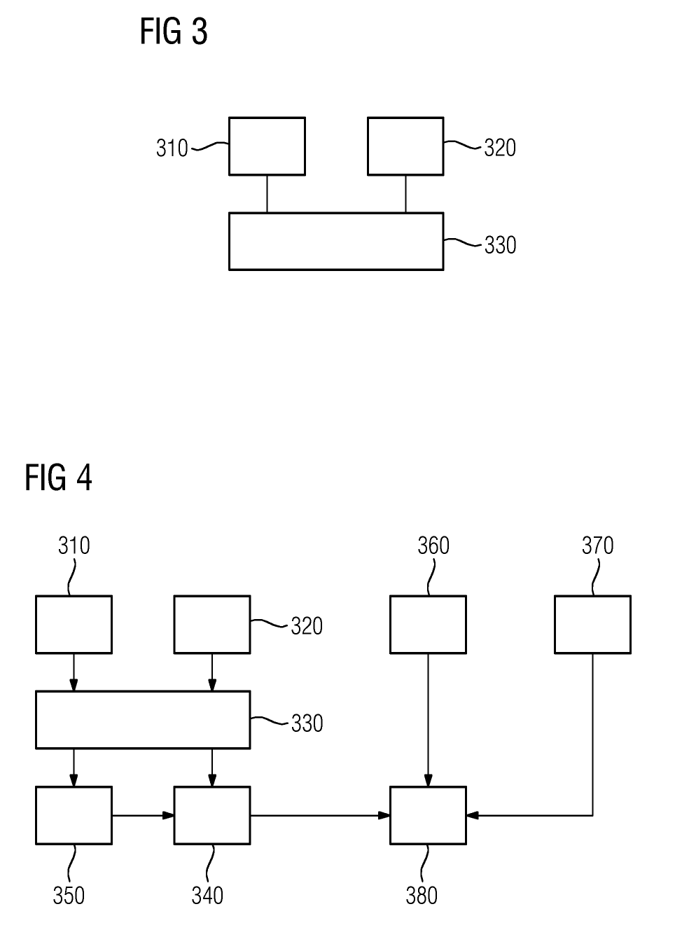

The magnetic resonance device 10 also has an intervention apparatus 100 that is positioned, for example, inside the patient 15. The intervention apparatus is connected to the evaluation unit 26 that is incorporated by the system control unit 22. The evaluation unit 26 is, among other things, capable of ascertaining an absolute temperature distribution using a reference temperature and a relative temperature distribution. For this purpose and other purposes that are illustrated in FIGS. 3 and 4, signals measured by the intervention apparatus (e.g., a reference temperature signal and/or a temperature distribution signal and/or a localization signal and/or an image data signal) are transmitted to the evaluation unit.

The system control unit 22 also includes appropriate software and/or computer programs that may be loaded in a memory of the system control unit 22, having program code to carry out the method for determining temperature when the program is run in the system control unit 22 of the magnetic resonance device 10.

The illustrated magnetic resonance device 10 in the present exemplary embodiment may include further components that magnetic resonance devices conventionally have. A general mode of operation of a magnetic resonance device 10 is known to a person skilled in the art.

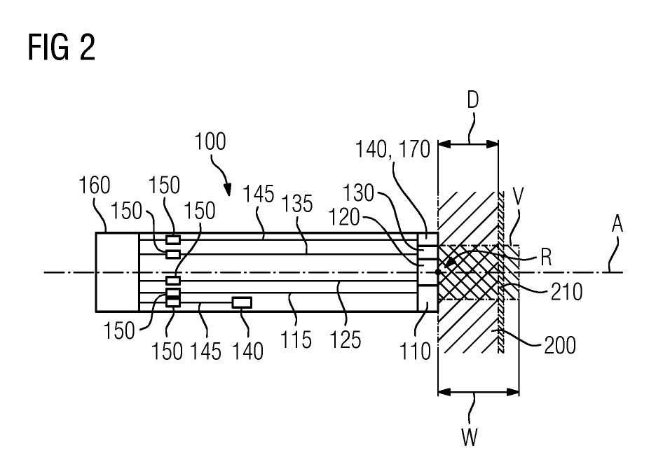

FIG. 2 schematically depicts an embodiment of an intervention apparatus 100. The intervention apparatus 100 includes an intervention unit 110, a reference temperature measuring unit 120 for detecting a reference temperature, and a temperature distribution measuring unit 130 for detecting a relative temperature distribution (e.g., units). The units 110, 120, 130 are each connected by supply lines 115, 125, 135, 145 to an interface 160. The interface 160 is configured to connect the intervention apparatus 100 to the evaluation unit 26 for transmitting signals. Possible signals are, for example, a control signal for controlling the intervention unit and/or a temperature distribution signal measured by the temperature distribution measuring unit 130 and/or a reference temperature signal measured by the reference temperature measuring unit 110 and/or a localization signal measured by the localization unit 140. The supply lines include blocking circuits 150 that suppress coupling of electrical currents (e.g., by the high-frequency antenna unit 20) into the intervention apparatus 100.

For example, the intervention unit 110 is arranged at the end, (e.g., at the tip) of the intervention apparatus. In this example, the intervention unit is an ablation unit, such as an HF electrode that is configured to carry out an ablation of an organic tissue 200. The organic tissue 200 is, for example, a myocardium having a thickness D. The tip of the intervention apparatus 100 rests on the myocardium 200 opposite the pericardium 210.

In the example, the temperature distribution measuring unit 130 includes an HF coil that is capable of receiving magnetic resonance signals. The temperature distribution measuring unit has a measuring volume V that has an extension W that is parallel to a longitudinal axis A of the intervention apparatus 100. The extension W is sufficiently sized to be able to receive scan data over the entire thickness W of the myocardium.

The reference temperature measuring unit 120 is configured to measure a reference temperature signal at a reference location R inside the patient, for example, using a thermoelement. An absolute temperature may be derived as the reference temperature signal in contrast to the temperature distribution signal.

The localization unit 140 includes, for example, two HF coils that are configured to receive HF signals (e.g., magnetic resonance signals). One of the two HF coils is arranged at the end at the tip of the intervention apparatus 100.

The magnetic resonance signals may be used to generate an image of at least part of the patient 15 in addition to tracking. In the example, the end HF coil 140 simultaneously constitutes an image data acquisition unit 170 (e.g., the image data acquisition unit 170 is incorporated by the localization unit 140). However, the intervention apparatus 100 may have a separate image data acquisition unit 170 for recording image data signals and may potentially be configured more specifically for image data acquisition.

In one embodiment, the temperature distribution measuring unit 130 may include the image data acquisition unit 170, and/or the temperature distribution measuring unit 130, the image data acquisition unit 170, and the localization unit 140 may be integrated in an HF coil.

FIG. 3 schematically depicts an embodiment of the method for determining temperature with the aid of the intervention apparatus 100 inside the examination object 15. At act 310, a reference temperature at a detection location R inside the examination object is detected using the reference temperature measuring unit 120. In the example shown in FIG. 2, the detection location R is located directly at the tip of the intervention apparatus 100.

At act 320, which takes place with a delay or at the same time as act 310, the relative temperature distribution in the measuring volume V, with a spatial position in relation to the detection location of the reference temperature that is known, is detected using a temperature distribution measuring unit 130. The detection location R is located inside the measuring volume V or immediately adjacent thereto since the detection location R facilitates the subsequent ascertainment of the absolute temperature distribution in act 330. The absolute temperature distribution is ascertained using the previously detected reference temperature and the relative temperature distribution via an evaluation unit.

A reference temperature signal measured by the reference temperature measuring unit 120 in act 310 is transmitted to the evaluation unit 26. A temperature distribution signal measured by the temperature distribution measuring unit 130 in act 320 is analogously also transmitted to the evaluation unit 26. The supply lines 125 and 135 shown in FIG. 2 and the interface 160 connected thereto may be used for transmitting the signals.

Various methods may be used for ascertaining the relative temperature distribution using the reference temperature signal measured in act 310, such as, for example, a measurement of a phase difference and/or a diffusion and/or a T1 relaxation and/or a T2 relaxation and/or a proton resonance frequency and/or a proton density. The magnetic resonance sequence that is irradiated by the high-frequency antenna unit 20 of the magnetic resonance device 10 into the patient 15 is adjusted accordingly.

FIG. 4 depicts an expanded variant of the method shown in FIG. 3. According to this, the ascertained absolute temperature distribution is provided in act 340. The provision may be made, for example, with the aid of the display unit 24 of the user interface 23 of the magnetic resonance device 10. A temperature map that represents temperature values incorporated, for example, using color coding of the absolute temperature distribution may be displayed. Higher temperature values may be displayed in reddish color tones, and lower temperature values may be displayed in bluish color tones.

If one or more temperature value(s) of the absolute temperature distribution exceeds a previously determined threshold value, a feedback signal may be output in act 350. The feedback signal may be output in conjunction with the display of the absolute temperature distribution in act 340 (e.g., by expressing temperature values that exceed the threshold value via flashing pixels). Further alternative and/or supplementary forms of the feedback signal such as, for example, acoustic or haptic signals may also be provided. Action may be taken in the control of the intervention unit 100 using the feedback signal (e.g., by stopping a possible ablation process).

The position of the intervention apparatus 100 inside the examination object 15 is advantageously also determined in act 360. As a result, an assessment and/or check as to at which location inside the examination object 15 the intervention apparatus 100 is positioned is and/or how the intervention apparatus 100 is set up may be made

The position determination may be made, for example, by the localization unit 140 shown in FIG. 2 measuring a localization signal that is transmitted to the evaluation unit 26. Transmission may occur using the supply line 145 and the interface 160 connected to the evaluation unit 26.

The localization signal may, for example, include information that may be used in the tracking method. HF signals may therefore be irradiated into the patient 15 (e.g., by the high-frequency antenna unit 20 of the magnetic resonance device 10). The HF signals may be spatially encoded by suitable wiring of the gradient coil unit 18 and be at least partially received as a localization signal by the localization unit 140. Since the arrangement of the localization unit 140 on the intervention apparatus 100 is known, the position of the intervention apparatus 100 may be determined using the received spatially encoded localization signals.

An image data signal may be detected in act 370. The image data signal may be transmitted to the evaluation unit 26. The image data signal may be detected, for example, by an image data acquisition unit 170 of the intervention apparatus 100. In the example depicted in FIG. 2, the image data acquisition unit 170 is incorporated by the localization unit 140. Magnetic resonance signals for imaging (e.g., image data signal) may be detected by the localization unit 140 in addition to magnetic resonance signals for position determination of the intervention apparatus 100 (e.g., localization signals). The signals may be the same, so the same signal is used for both purposes. Localization signals and image data signal may be different, however, and, for example, be measured alternately one after the other by the combined localization unit 140 and image data acquisition unit 170. The image data signal may be transmitted via the supply line 145 and the interface 160 to the evaluation unit 26.

The localization unit 140 and the image data acquisition unit 170 may also be separate units having, for example, separate supply lines.

The ascertained absolute temperature distribution and/or the localized position of the intervention apparatus 100 and/or the generated image of at least part of the examination object 15 may advantageously be provided jointly in act 380, for example, in that the ascertained absolute temperature distribution and/or the localized position of the intervention apparatus 100 and/or the generated image are displayed using the display unit 24. The display may be such that the generated image in which the localized position of the intervention apparatus 100 and/or a temperature map based on the absolute temperature distribution is displayed in the correct position is reproduced. A person involved in an intervention may therefore effectively observe and control the intervention.

During detection of the reference temperature 310 and the relative temperature distribution 320, the intervention apparatus 100 may rest on a moving organ, such as, for example, a myocardium 200. A potentially disruptive relative movement between measuring object and measuring apparatus may be suppressed thereby.

To summarize, certain embodiments enable effective observation of an intervention. For example, the success of an ablation and control of an ablation may be determined in real time by temperature measurement. For effective observation, the temperature is locally determined in a relatively small volume (e.g., in the myocardium cross-section adjoining the tip of an intervention apparatus).

Reference is again made to the fact that the methods and devices described in detail above are merely exemplary embodiments that may be modified in a wide variety of ways by a person skilled in the art without departing from the scope of the invention. Use of the indefinite article “a” or “an” does not prevent the relevant features from also being present multiple times. Similarly, the term “unit” does not prevent the relevant components from including a plurality of interacting sub-components that may optionally also be spatially distributed.

It is to be understood that the elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent, and that such new combinations are to be understood as forming a part of the present specification.

While the present invention has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. A method for determining temperature with the aid of an intervention apparatus inside an examination object, the method comprising:
detecting, by a reference temperature measuring unit, a reference temperature at a detection location inside the examination object;
detecting, by a temperature distribution measuring unit, a relative temperature distribution in a measuring volume, wherein a spatial position of the measuring volume in relation to the detection location of the reference temperature is known; and
ascertaining, by a processor, an absolute temperature distribution using the reference temperature and the relative temperature distribution.

2. The method of claim 1, wherein detecting the reference temperature comprises measuring, by the reference temperature measuring unit of the intervention apparatus, a reference temperature signal at the reference location inside the examination object, and
wherein the method further comprises transmitting the measured reference temperature signal to the processor.

3. The method of claim 1, wherein detecting the relative temperature distribution comprises measuring, by the temperature distribution measuring unit of the intervention apparatus, a temperature distribution signal from the measuring volume inside the examination object, and wherein the method further comprises transmitting the measured temperature distribution signal to the processor.

4. The method of claim 1, wherein detecting the relative temperature distribution comprises measuring t a phase difference, a diffusion, a T1 relaxation, a T2 relaxation, a proton resonance frequency, a proton density, or any combination thereof.

5. The method of claim 1, further comprising:
displaying the ascertained absolute temperature distribution.

6. The method of claim 1, further comprising:
outputting a feedback signal in the presence of a feedback condition as a function of the ascertained absolute temperature distribution.

7. The method of claim 1, further comprising:
determining a position of the intervention apparatus inside the examination object.

8. The method of in claim 7, wherein determining the position of the intervention apparatus comprises
measuring, by a localization unit of the intervention apparatus, a localization signal, and wherein the method further comprises transmitting the localization signal to the processor.

9. The method of claim 1, further comprising:
generating an image of at least part of the examination object.

10. The method of claim 9, wherein generating the image comprises:
detecting, by an image data acquisition unit of the intervention apparatus, a further apparatus, or a combination thereof, an image data signal, and wherein the method further comprises transmitting the image data signal to the processor.

11. The method of claim 9, further comprising:
displaying the ascertained absolute temperature distribution, a localized position of the intervention apparatus, the generated image of at least part of the examination object, or any combination thereof jointly.

12. The method of claim 1, wherein during detection of the reference temperature and the relative temperature distribution, the intervention apparatus rests on a moving organ.

13. An intervention apparatus comprising:
an intervention unit;
a reference temperature measuring unit configured to detect a reference temperature signal; and
a temperature distribution measuring unit configured to detect a temperature distribution signal.

14. The intervention apparatus of claim 13, wherein the intervention unit is configured to carry out an ablation of an organic tissue.

15. The intervention apparatus of claim 13, wherein the intervention unit is arranged at an end of the intervention apparatus.

16. The intervention apparatus of claim 13, wherein the temperature distribution measuring unit comprises at least one HF coil.

17. The intervention apparatus of claim 13, wherein the temperature distribution measuring unit comprises a measuring volume,
wherein a temperature at a reference location, that is located inside the measuring volume, is ascertainable using the detected reference temperature signal.

18. The intervention apparatus of claim 13, wherein the temperature distribution measuring unit has a measuring volume that comprises an extension of a myocardial thickness parallel to a longitudinal axis of the intervention apparatus.

19. The intervention apparatus of claim 13, wherein the reference temperature measuring unit comprises a temperature sensor.

20. The intervention apparatus of claim 13, further comprising:
a localization unit.

21. The intervention apparatus of claim 20, wherein the localization unit comprises at least one HF coil configured to receive an HF signal.

22. The intervention apparatus of claim 13, further comprising:
an image data acquisition unit.

23. The intervention apparatus of claim 13, further comprising:
at least one supply line for the intervention unit, the reference temperature measuring unit, and the temperature distribution measuring unit, the at least one supply line comprising blocking circuits.

24. The intervention apparatus of claim 13, further comprising:
an interface configured to connect the intervention apparatus to an evaluation unit for transmitting signals.

25. A magnetic resonance device comprising:
an intervention unit,
a reference temperature measuring unit configured to detect a reference temperature signal,
a temperature distribution measuring unit configured to detect a temperature distribution signal.
a processor in communication with the reference temperature measuring unit and the temperature distribution measuring unit.

26. The magnetic resonance device of claim 25, wherein the processor is configured to ascertain an absolute temperature distribution using the reference temperature signal and the temperature distribution signal.

27. In a non-transitory computer-readable storage medium that stores instructions executable by a programmable system controller of a magnetic resonance device to determine temperature, the instructions comprising:
detecting, by a reference temperature measuring unit, a reference temperature at a detection location inside the examination object;
detecting, by a temperature distribution measuring unit, a relative temperature distribution in a measuring volume, wherein a spatial position of the measure volume in relation to the detection location of the reference temperature is known; and
ascertaining by an evaluation unit, an absolute temperature distribution using the reference temperature and the relative temperature distribution.
